(19) United States
(12) Patent Application Publication
(10) Pub. No.: US 2025/0287984 A1
(43) Pub. Date: Sep. 18, 2025

(54) INFANT FORMULA FOR IMPROVING COGNITIVE DEVELOPMENT

(71) Applicant: N. V. NUTRICIA, Zoetermeer (NL)

(72) Inventors: Anniek Lidewij SCHIPPER, Utrecht (NL); Noela Eliza SCHAAP, Utrecht (NL)

(73) Assignee: N. V. NUTRICIA, Zoetermeer (NL)

(21) Appl. No.: 19/227,532

(22) Filed: Jun. 4, 2025

Related U.S. Application Data
(63) Continuation of application No. PCT/EP2023/084505, filed on Dec. 6, 2023.

(30) Foreign Application Priority Data
Dec. 6, 2022 (EP) 22211769.9

(51) Int. Cl. A23L 33/00; A23L 33/12; A23L 33/125; A23L 33/17

(52) U.S. Cl. CPC A23L 33/40; A23L 33/12; A23L 33/125; A23L 33/17

(57) ABSTRACT

A nutritional composition comprising digestible carbohydrates, protein and lipid, wherein the lipid is in the form of lipid globules which are at least partly coated on the surface with phospholipids, for increasing myelination in the brain of an infant who has delayed brain development or is at risk of having delayed brain development.

### FIELD OF THE INVENTION

The present invention relates to a nutritional composition for an infant who has delayed brain development or is at risk of having delayed brain development.

### BACKGROUND OF THE INVENTION

Human milk is the uncontested gold standard concerning infant nutrition. However, in some cases breastfeeding is inadequate or unsuccessful for medical reasons or because of a choice not to breastfeed. For such situations infant or follow-on formulas have been developed. Commercial infant formulas are commonly used today to provide supplemental or sole source of nutrition early in life. These formulas comprise a range of nutrients to meet the nutritional needs of the growing infant, and typically include fat, carbohydrate, protein, vitamins, minerals, and other nutrients helpful for optimal infant growth and development. Commercial infant formulas are designed to mimic, as closely as possible, the composition and function of human milk.

The decision to breastfeed is an early parental decision that may affect a child's later cognitive and behavioural functioning. The prevailing consensus from large-scale epidemiological studies is that children who were breastfed perform, on average, higher on tests of IQ and cognitive functioning than do children who were exclusively formula-fed, even when factors such as birth-weight, gestation duration, and maternal education and socioeconomic status are accounted for.

These neuropsychological findings are complemented by morphometric brain imaging studies in adolescents, showing volumetric increases in total white matter, sub-cortical grey matter and parietal lobe cortical thickness in those who were breastfed as infants. Non-imaging assessments of early neural pathway maturation using evoked potentials have further supported the developmental benefits of breastfeeding, with formula-fed infants found to have greater wave latencies in their visual and auditory pathways at 1 year of age (Khedr et al., 2004, DOI: 10.1111/j.1651-2227.2004.tb03011.x), suggestive of delayed or immature myelination of these pathways, compared to breastfed infants.

The primary hypothesized substrate for these developmental advantages is the rich compliment of long-chain fatty acids found in breast milk, specifically docosahexaenoic (DHA) and arachidonic (AA) acids (McCann and Ames, 2005, DOI: 10.1093/ajcn.82.2.281). Together, DHA and AA comprise approximately 20% of the fatty acid content of the brain and are involved in early neurodevelopment by promoting healthy neuronal growth, repair, and myelination (Guesnet and Alessandri, 2011, DOI: 10.1016/j.biochi.2010.05.005). Optimal foetal neurodevelopment is amongst others dependent on DHA. DHA deficiency leads to reduced dendritic arborisation and impaired expression of the genes that regulate neurogenesis, neurotransmission, and connectivity.

Myelin is a lipid-rich material that surrounds nerve cell axons to insulate them and increase the rate at which electrical impulses (called action potentials) are passed along the axon. Myelin is formed in the central nervous system (CNS; brain, spinal cord and optic nerve) by glial cells called oligodendrocytes and in the peripheral nervous system (PNS) by glial cells called Schwann cells. In the CNS, axons carry electrical signals from one nerve cell body to another. In the PNS, axons carry signals to muscles and glands or from sensory organs such as the skin.

This “insulating” role for myelin is essential for normal motor function (i.e. movement such as walking), sensory function (e.g. hearing, seeing or feeling the sensation of pain) and cognition (e.g. acquiring and recalling knowledge), as demonstrated by the consequences of disorders that affect it, such as the genetically determined leukodystrophies; acquired inflammatory demyelinating disorder, multiple sclerosis; and inflammatory demyelinating peripheral neuropathies.

The process of generating myelin is called myelination or myelinogenesis. In humans, myelination begins early in the 3rd trimester of pregnancy, although only little myelin is present in either the CNS or the PNS at the time of birth. During infancy, myelination progresses rapidly, with increasing numbers of axons acquiring myelin sheaths. This corresponds with the development of cognitive and motor skills, including language comprehension, speech acquisition, crawling and walking. Myelination continues through adolescence and early adulthood and although largely complete at this time, myelin sheaths can be added in grey matter regions such as the cerebral cortex, throughout life.

Deoni et al., 2018, doi:10.1016/j.neuroimage.2017.12.056, describes an examination of longitudinal trajectories of brain and neurocognitive development in children who were exclusively breastfed versus formula-fed for at least 3 months. They further examined development between children who received different formula compositions. Results revealed significantly improved overall myelination in breastfed children accompanied by increased general, verbal, and non-verbal cognitive abilities compared to children who were exclusively formula-fed. These differences were found to persist into childhood even with groups matched for important socioeconomic and demographic factors. They also found significant developmental differences depending on formula composition received and that, in particular, long-chain fatty acids, iron, choline, sphingomyelin and folic acid are significantly associated with early myelination trajectories.

WO2017/102720 describes a nutritional composition for infants and young children comprising a phospholipid, a metabolic precursor and/or metabolite thereof. The phospholipid promotes and/or supports an optimal myelination trajectory in the brain, such trajectory being close to that observed in infants fed exclusively with human breast milk.

Oshida et al., 2003, DOI: 10.1203/01.PDR.0000054654.73826.AC describes that dietary sphingomyelin (SM) contributes to CNS (central nervous system) myelination in developing rats in whom the sphingolipid biosynthesis was chemically blocked by L-Cycloserine.

Tanaka et al., 2013, http://dx.doi.org/10.1016/j.braindev.2012.03.004 describes a randomised control trial to examine the effects of sphingomyelin (SM), on the mental, motor and behavioral development of premature infants. They report that nutritional intervention via administration of SM-fortified milk has a positive association with the neurobehavioral development of low-birth-weight infants.

Human milk lipids are known to have a distinct physical structure composed of large lipid globules with a mode diameter, based on volume, of about 4 μm existing of a triglyceride core coated by a tri-layer of membranes, the milk fat globule membrane (MFGM). The mode diameter, based on volume, of lipid droplets in standard infant formula is typically about 0.3-0.5 μm due to the industrial processing procedures applied to achieve stable products, and the lipid droplets are not surrounded by MFGM but mostly by milk proteins. Infant formula with lipid globules with an architecture more similar to the lipid globules in human milk have been described.

WO2011/115490 describes a nutritional composition comprising phospholipids for use in altering the brain membrane fatty acid composition and for the amelioration of cognitive performance and behavioral performance in a human subject.

WO2011/115491 describes a nutritional composition comprising lipid globules with a defined particle size distribution for use in altering the brain membrane fatty acid composition and for the amelioration of cognitive performance and behavioral performance in a human subject.

WO2018/104512 describes a nutritional composition with lipid globules with a defined particle size distribution and further comprising an increased level of sn-2 palmitate for altering the cell membrane fatty acid composition, in particular in brain and red blood cell membranes, and for improving cognitive development and behavioral performance in a human subject.

### SUMMARY OF THE INVENTION

The inventors of the present invention have surprisingly found that, besides the presence of sphingomyelin or specific fatty acids such as DHA and ARA in nutritional compositions for infants and young children, also the supramolecular lipid structure in the nutritional composition affects the rate of myelination in an infant who has delayed brain development or is at risk of having delayed brain development.

Accordingly the invention pertains to a nutritional composition, selected from infant formula, follow-on formula and young child formula, comprising digestible carbohydrates, protein and lipid, wherein the lipid is in the form of lipid globules and wherein
a. the lipid globules have a mode diameter, based on volume, of at least 1.0 μm; and/or
b. at least 45 volume %, based on total lipid volume, of the lipid globules have a diameter of 2 to 12 μm; and
c. the lipid comprises 0.5 to 20 wt. % phospholipids based on total lipids and wherein the lipid globules are at least partly coated on the surface with phospholipids,
   for use in increasing myelination in the brain of an infant who has delayed brain development or is at risk of having delayed brain development.

The inventors performed an experiment wherein the mice dams were exposed to an omega-3 fatty acid deficient diet during pregnancy and lactation, resulting in impaired brain development in the offspring (Basak et al., 2020, doi:10.3390/nu12123615). Subsequently the offspring was fed with two different diets. The Control diet comprised small lipid droplets and phospholipids added in dry-blended form (i.e. not present in the coating). The Test diet comprised large, phospholipid coated lipid globules. Besides these differences the Control and Test diet were exactly the same. The offspring received the Control or Test diet from post-natal day 16 till 42. The fatty acid content was determined In the brains of the offspring. In the hippocampal region the gene expression (qPCR) of Myelin Basic Protein (MBP) and Myelin-Associated Glycoprotein (MAG), which are markers for myelin, was determined.

In the offspring group that consumed the Test diet the brain cell membrane levels of palmitic acid (C16:0) and stearic acid (C18:0) were increased compared to the levels observed in the offspring group that consumed the Control diet. C16:0 and C18:0 are the two most abundant saturated fatty acids in the brain early in life [Martínez et al., 1998, doi:10.1046/j.1471-4159.1998.71062528.x.]. Myelin is particularly rich in these fatty acids [Manzoli et al., 1970, doi:10.1016/0014-5793(70)80462-8].

In the offspring group that consumed the Test diet the levels of MBP and MAG in the hippocampal region were increased compared to the levels observed in the offspring group that consumed the Control diet. The hippocampus is an important area for brain function that matures relatively late. The myelination pattern of the hippocampus is critical for brain function. Myelination of hippocampus is ongoing during the diet intervention period, see Nickel et al., 2018, doi:10.1155/2018/6436453.

The effects of dietary treatment with the nutritional composition comprising large, phospholipid coated lipid droplets is unexpected. While it is known that fatty acid composition of diet and in particular the addition of sphingomyelin to diet may influence brain fatty acid status, myelination and cognitive function, the fatty acid composition and the sphingomyelin levels were exactly the same in both the Control and Test diet. The additional increase in myelin in the brain in the Test group may therefor be linked to the phospholipid being present in the coating and the larger size of the lipid globules.

### DETAILED DESCRIPTION OF THE INVENTION

A first aspect of the invention relates to a nutritional composition, selected from infant formula, follow-on formula and young child formula, comprising digestible carbohydrates, protein and lipid, wherein the lipid is in the form of lipid globules and wherein
a. the lipid globules have a mode diameter, based on volume, of at least 1.0 μm; and/or
b. at least 45 volume %, based on total lipid volume, of the lipid globules have a diameter of 2 to 12 μm; and
c. the lipid comprises 0.5 to 20 wt. % phospholipids based on total lipids and wherein the lipid globules are at least partly coated on the surface with phospholipids,
   for use in increasing myelination in the brain of an infant who has delayed brain development or is at risk of having delayed brain development.

For some jurisdictions, the invention may also be worded as a method for increasing myelination in the brain of an infant who has delayed brain development or is at risk of having delayed brain development, said method comprising feeding said infant a nutritional composition, selected from infant formula, follow-on formula and young child formula, comprising digestible carbohydrates, protein and lipid, wherein the lipid is in the form of lipid globules and wherein
a. the lipid globules have a mode diameter, based on volume, of at least 1.0 μm; and/or
b. at least 45 volume %, based on total lipid volume, of the lipid globules have a diameter of 2 to 12 μm; and
c. the lipid comprises 0.5 to 20 wt. % phospholipids based on total lipids and wherein the lipid globules are at least partly coated on the surface with phospholipids.

For some jurisdictions, the invention may also be worded as the use of digestible carbohydrates, protein and lipid in the manufacture of a nutritional composition for increasing myelination in the brain of an infant who has delayed brain development or is at risk of having delayed brain development, wherein the nutritional composition, selected from infant formula, follow-on formula and young child formula, comprising digestible carbohydrates, protein and lipid, wherein the lipid is in the form of lipid globules and wherein
a. the lipid globules have a mode diameter, based on volume, of at least 1.0 μm; and/or
b. at least 45 volume %, based on total lipid volume, of the lipid globules have a diameter of 2 to 12 μm; and
c. the lipid comprises 0.5 to 20 wt. % phospholipids based on total lipids and wherein the lipid globules are at least partly coated on the surface with phospholipids.

In some jurisdictions administering a nutritional composition to an infant is considered non-therapeutic. In those instances the invention may be worded as defined above by way of a method comprising administering a nutritional composition. For clarity, the method can also be defined as a non-therapeutic method. By definition, the words “non-therapeutic” exclude any therapeutic effect.

### Application

The “myelination level in the brain of an infant” may suitably determined in a MRI scanner.

The term “infant” as used herein refers to a child aged 0-36 months, preferably to a child aged 0-24 months, more preferably to a child aged 0-12 months, and most preferably to a child aged 0-6 months. Preferably, the infant is a human infant.

Brain development of the infant may be suitably assessed with the Bayley III Test. This test refers to the Bayley Scales of Infant Development version 3 (BSID-III), which is a standard series of measurements used primarily to assess the motor (fine and gross), language (receptive and expressive), and cognitive development of infants and toddlers at ages 16 days till 42 months. The primary purpose of the Bayley-III is to identify suspected developmental delays in children through the use of norm-referenced scores. (Bayley N. Bayley Scales of Infant and Toddler Development. 3rd edn. San Antonio, TX: Harcourt Assessment Inc, 2006).

In a preferred embodiment, the delayed brain development in the infant who has delayed brain development is treated or reduced by the increased myelination.

In another preferred embodiment, the risk of having delayed brain development is reduced by the increased myelination for the infant at risk of having delayed brain development. In other words, preferably delayed brain development is prevented by increased myelination in the infant at risk of having delayed brain development.

Preferably, the increased myelination contributes to improved cognitive development and/or improved motor function in the infant.

Preferably, the increased myelination is around the axons connected to and within the hippocampus.

Preferably, the increased myelination is by higher levels in the brain of myelin-associated glycoprotein (MAG), myelin basic protein (MBP), or combinations thereof. More preferably, the increased myelination is by higher levels of MAG, MBP, or combinations thereof in the hippocampal region of the brain.

Preferably, the increased myelination is by increasing the brain cell membrane levels of palmitic acid (C16:0), stearic acid (C18:0), or combination thereof.

Preferably, the increased myelination in the brain of the infant is compared to a similar infant who has delayed brain development or is at risk of having delayed brain development, which consumed a nutritional composition, selected from infant formula, follow-on formula and young child formula, comprising digestible carbohydrates, protein and lipid, and wherein the lipid is in the form of lipid globules and wherein:
a. the lipid globules have a mode diameter, based on volume, of around 0.5 μm, and
b. less than 45 volume %, based on total lipid volume, of the lipid globules have a diameter above 2 μm, and
c. wherein the lipid comprises less than 0.5 wt. % phospholipids based on total lipids and wherein the lipid globules are not coated with phospholipids.

Preferably, the infant who has delayed brain development or is at risk of having delayed brain development is selected from preterm infants, small-for-gestational age infants, infants with phenylketonuria (PKU), infants with epilepsy, infants with cerebral palsy, infants born with a brain injury, infants who were exposed to perinatal hypoxia, or combinations thereof.

In one preferred embodiment, the infant is selected from preterm infants, small-for-gestational age infants or combinations thereof. The term “preterm infant” as used herein refers to a subject born before 37 weeks of gestational age. Preterm infants are at greater risk for cerebral palsy, delays in development, hearing problems, and problems seeing. These risks are greater the earlier a baby is born. The term “small for gestational age infant” as used herein refers to an infant born smaller in size than normal for their gestational age, most commonly defined as a weight below the 10th percentile for their gestational age.

In another preferred embodiment, the infant is term born infant who is not small-for-gestational age, and wherein the infant is in addition one or more of infants with phenylketonuria (PKU) infants with epilepsy, infants with cerebral palsy, infants born with a brain injury, infants who were exposed to perinatal hypoxia.

### Lipid

The nutritional composition for use according to the present invention comprises lipid. Lipid in the present invention comprises one or more selected from the group consisting of triglycerides, polar lipids (such as phospholipids, cholesterol, glycolipids, sphingomyelin), free fatty acids, monoglycerides and diglycerides. Preferably the composition comprises at least 70 wt. %, more preferably at least 80 wt. %, even more preferably at least 85 wt. % triglycerides, most preferably at least 90 wt. % triglycerides based on total lipid.

The lipid provides preferably 30 to 60% of the total calories of the nutritional composition. More preferably the nutritional composition comprises lipid providing 35 to 55% of the total calories, even more preferably the nutritional composition comprises lipid providing 40 to 50% of the total calories. The lipid is preferably present in an amount of 3 to 7 g per 100 kcal, more preferably in an amount of 4 to 6 g lipid per 100 kcal and most preferably in an amount of 4.5 to 5.5 g lipid per 100 kcal. When in liquid form, e.g. as a ready-to-feed liquid, the nutritional composition preferably comprises 2.1 to 6.5 g lipid per 100 ml, more preferably 3.0 to 4.0 g per 100 ml. Based on dry weight, the nutritional composition preferably comprises 10 to 50 wt. %, more preferably 12.5 to 40 wt. % lipid, even more preferably 19 to 30 wt. % lipid.

The lipid preferably comprises vegetable lipid. The presence of vegetable lipid advantageously enables an optimal fatty acid profile high in polyunsaturated fatty acids and/or more reminiscent to human milk fat. Lipid from non-human mammalian milk alone, e.g. cow milk, does not provide an optimal fatty acid profile. The amount of essential fatty acids is too low in non-human mammalian milk.

Preferably the nutritional composition comprises at least one, preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, palm oil and palm kernel oil.

In a preferred embodiment, the nutritional composition comprises 30 to 90 wt. % vegetable lipid based on total lipid, more preferably 35 to 80 wt. %, more preferably 40 to 70 wt. %, more preferably 40 to 60 wt. % vegetable lipid based on total lipid.

The lipid in the nutritional composition preferably further comprises mammalian milk fat, preferably ruminants milk fat, more preferably the mammalian milk fat is derived from cow milk, goat milk, sheep milk, buffalo milk, yak milk, reindeer milk, and/or camel milk, most preferably the mammalian milk fat is cow milk fat. Preferably the mammalian milk fat is not human milk fat. Preferably the mammalian milk fat comprises at least 70 wt. % triglycerides, more preferably at least 90 wt. %, more preferably at least 97 wt. % triglycerides by weight of the mammalian milk fat.

Preferably the mammalian milk fat is derived from butter, butter fat, butter oil, and/or anhydrous milk fat, more preferably the mammalian milk fat is derived from anhydrous milk fat and/or butter oil. Such mammalian milk fat sources are high in triglyceride levels. These mammalian milk fat sources may be in the form of a continuous lipid phase or a water-in-oil emulsion. The use of these mammalian milk fat sources during the manufacture of the nutritional composition of the present invention enables the formation of lipid globules, wherein each globule comprises a mixture of vegetable fat and mammalian milk fat.

Mammalian milk fat in the present invention refers to all lipid components of milk, as produced by the mammalians, such as the cow, and is found in commercial milk and milk-derived products. Butter in the present invention is a water-in-oil emulsion comprised of over 80 wt. % milk fat. Butterfat in the present invention relates to all of the fat components in milk that are separable by churning, in other words, present in butter. Anhydrous milk fat (AMF) is a term known in the art and relates to extracted milk fat. Typically AMF comprises more than 99 wt. % lipid based on total weight. It can be prepared from extracting milk fat from cream or butter. Anhydrous butter oil in the present invention is synonymous with AMF. Butter oil also is a term known in the art. It typically relates to a milk fat extract with more than 98 wt. % lipid and typically is a precursor in the process of preparing anhydrous milk fat or anhydrous butter oil.

Preferably the composition comprises 10 to 70 wt. % mammalian milk fat based on total lipid, more preferably 20 to 65 wt. %, more preferably 30 to 60 wt. %, more preferably 40 to 60 wt. % based on total lipid.

Preferably the ratio of vegetable fat to mammalian milk fat ranges from 3/7 to 9/1. In a preferred embodiment, the lipid in the nutritional composition comprises:
a) 35 to 80 wt. % vegetable lipid based on total lipid, and
b) 20 to 65 wt. % mammalian milk fat based on total lipid, wherein the mammalian milk fat is selected from butter, butter fat, butter oil or anhydrous milk fat.

More preferably, the lipid in the nutritional composition comprises:
a) 40 to 70 wt. % vegetable lipid based on total lipid, and
b) 30 to 60 wt. % mammalian milk fat based on total lipid, wherein the mammalian milk fat is selected from butter, butter fat, butter oil or anhydrous milk fat.

Most preferably, the lipid in the nutritional composition comprises:
a) 40 to 60 wt. % vegetable lipid based on total lipid, and
b) 40 to 60 wt. % mammalian milk fat based on total lipid, wherein the mammalian milk fat is selected from butter, butter fat, butter oil or anhydrous milk fat.

Compared to vegetable fat, mammalian milk fat is known to have a higher content of palmitic acid (PA) at the sn-2 position of a triglyceride. In a preferred embodiment, the lipid in the nutritional composition comprises at least 10 wt. % PA based on total fatty acids and at least 15 wt. % of PA, based on total palmitic acid, is located at the sn-2 position of a triglyceride. Preferably, the amount of PA is below 30 wt. % based on total fatty acids. More preferably, the amount of PA is from 12 to 26 wt. % based on total fatty acids, even more preferably from 14 to 24 wt. %.

Preferably, at least 15 wt. % PA, more preferably at least 20 wt. % PA, even more preferably at least 25 wt. % PA, most preferably at least 30 wt. % PA, based on total PA is in the sn-2 or beta position in a triglyceride. Preferably the amount of PA in the sn-2 position in a triglyceride is not more than 45 wt. %, preferably not more than 40 wt. % based on total PA present in the lipid. Preferably the amount of PA in the sn-2 position in a triglyceride is from 25 to 40 wt. % based on total PA.

Compared to vegetable fat, mammalian milk fat is known to have a higher content of short-chain fatty acids (SCFA) butyric acid (BA; C4:0) and caproic acid (CA; C6:0). In a preferred embodiment, the lipid in the nutritional composition comprises 0.6 to 5 wt. % SCFA being the sum of BA and CA based on total fatty acids. Preferably the nutritional composition comprises less than 5 wt. % BA based on total fatty acids, preferably less than 4 wt. %. Preferably the nutritional composition comprises at least 0.5 wt. % BA based on total fatty acids, preferably at least 0.6 wt. %, preferably at least 0.9 wt. %, more preferably at least 1.2 wt. % BA based on total fatty acids.

In a preferred embodiment, the lipid in the nutritional composition comprises:
at least 10 wt. % PA based on total fatty acids and at least 15 wt. % of PA, based on total PA, is located at the sn-2 position of a triglyceride; and.
0.6 to 5 wt. % SCFA being the sum of BA and CA based on total fatty acids.

The nutritional composition preferably also comprises one or more lipids selected from fish oil, egg lipid, and microbial, algal, fungal or single cell oils.

### Fatty Acid Composition

SFA relates to saturated fatty acids and/or acyl chains, MUFA relates to mono-unsaturated fatty acid and/or acyl chains, PUFA refers to polyunsaturated fatty acids and/or acyl chains with 2 or more unsaturated bonds; LC-PUFA refers to long chain polyunsaturated fatty acids and/or acyl chains comprising at least 20 carbon atoms in the fatty acyl chain and with 2 or more unsaturated bonds; Medium chain fatty acids (MCFA) refer to fatty acids and/or acyl chains with a chain length of 6, 8 or 10 carbon atoms. n3 or omega-3 PUFA refers to polyunsaturated fatty acids and/or acyl chains with 2 or more unsaturated bonds with an unsaturated bond at the third carbon atom from the methyl end of the fatty acyl chain, n6 or omega-6 PUFA refers to polyunsaturated fatty acids and/or acyl chains with 2 or more unsaturated bonds with an unsaturated bond at the sixth carbon atom from the methyl end of the fatty acyl chain.

In the context of the present invention, a weight percentage of fatty acids based on total fatty acids is calculated as if all fatty acids are free fatty acids, hence it is not taken into account whether a fatty acid is attached to a glycerol backbone or not.

DHA refers to docosahexaenoic acid and/or acyl chain (22:6 n3); DPA refers to docosapentaenoic acid and/or acyl chain (22:5 n3); n6 DPA refers to omega-6 docosapentaenoic acid and/or acyl chain (22:5 n6). EPA refers to eicosapentaenoic acid and/or acyl chain (20:5 n3); ARA refers to arachidonic acid and/or acyl chain (20:4 n6). LA refers to linoleic acid and/or acyl chain (18:2 n6); ALA refers to alpha-linolenic acid and/or acyl chain (18:3 n3).

LA refers to linoleic acid and/or acyl chain and is an n6 PUFA (18:2 n6) and the precursor of n6 LC-PUFA and is an essential fatty acid as it cannot be synthesized by the human body. The nutritional composition preferably comprises LA. LA preferably is present in a sufficient amount to promote a healthy growth and development, yet in an amount as low as possible to prevent negative, competitive, effects on the formation of n3 PUFA and a too high n6/n3 ratio. The nutritional composition therefore preferably comprises less than 20 wt. % LA based on total fatty acids, preferably 5 to 16 wt. %, more preferably 10 to 14.5 wt. %. Preferably, the nutritional composition comprises at least 5 wt. % LA based on total fatty acids, preferably at least 6 wt. % LA, more preferably at least 7 wt. % LA based on total fatty acids. Per 100 kcal, the nutritional composition preferably comprises 350-1400 mg LA.

ALA refers to alpha-linolenic acid and/or acyl chain and is an n3 PUFA (18:3 n3) and the precursor of n3 LC-PUFA and is an essential fatty acid as it cannot be synthesized by the human body. The nutritional composition preferably comprises ALA. Preferably ALA is present in a sufficient amount to promote a healthy growth and development of the infant. The nutritional composition preferably comprises at least 1.0 wt. %, more preferably the nutritional composition comprises at least 1.5 wt. %, even more preferably at least 2.0 wt. % ALA based on total fatty acids. Preferably the nutritional composition comprises less than 10 wt. % ALA, more preferably less than 5.0 wt. %, based on total fatty acids.

Preferably the nutritional composition comprises a weight ratio of LA/ALA from 2 to 20, more preferably from 3 to 16, even more preferably from 4 to 14, most preferably from 5 to 12.

The lipid in the nutritional composition preferably comprises 5 to 35 wt. % PUFA, based on total fatty acids, comprising LA and ALA in a weight ratio LA/ALA of 2 to 20.

Preferably, the nutritional composition comprises n3 LC-PUFA, such as EPA, DPA and/or DHA, more preferably DHA. As the conversion of ALA to DHA may be less efficient in infants, preferably both ALA and DHA are present in the nutritional composition. Preferably the nutritional composition comprises at least 0.05 wt. %, preferably at least 0.1 wt. %, more preferably at least 0.2 wt. %, of DHA based on total fatty acids. Preferably the nutritional composition comprises not more than 2.0 wt. %, preferably not more than 1.0 wt. % of DHA based on total fatty acids.

The nutritional composition preferably comprises ARA. Preferably the nutritional composition comprises at least 0.05 wt. %, preferably at least 0.1 wt. %, more preferably at least 0.2 wt. % of ARA based on total fatty acids. As the group of n6 fatty acids, especially arachidonic acid (ARA) counteracts the group of n3 fatty acids, especially DHA, the nutritional composition preferably comprises relatively low amounts of ARA. Preferably the nutritional composition comprises not more than 2.0 wt. %, preferably not more than 1.0 wt. % of ARA based on total fatty acids. Preferably the weight ratio between DHA and ARA is between 1:4 to 4:1, more preferably between 1:2 to 2:1, more preferably between 0.6 and 1.5.

### Lipid Globule Size

The lipid in the nutritional composition is in the form of lipid globules, wherein:
a. the lipid globules have a mode diameter, based on volume, of at least 1.0 μm; and/or
b. at least 45 volume %, based on total lipid volume, of the lipid globules have a diameter of 2 to 12 μm.

Lipid is typically present in the nutritional composition in the form of lipid globules. When the nutritional composition is in liquid form, these lipid globules are emulsified in the aqueous phase. Alternatively, when the nutritional composition is in powder form, the lipid globules are present in the powder and the powder is suitable for reconstitution with water or another food grade aqueous phase. The lipid globules may comprise a core and a surface.

The lipid globules in the nutritional composition preferably have mode diameter, based on volume, of at least 1.0 μm, more preferably at least 2.0 μm, and most preferably at least 3.0 μm. Preferably, the lipid globules have a mode diameter, based on volume, between 1.0 and 10 μm, more preferably between 2.0 and 8.0 μm, even more preferably between 3.0 and 7.0 μm, and most preferably between 4.0 μm and 6.0 μm.

Alternatively, or preferably in addition, the size distribution of the lipid globules is preferably in such a way that at least 45 volume % (vol. %), preferably at least 55 vol. %, even more preferably at least 65 vol. %, and most preferably at least 75 vol. %, based on total lipid volume, of the lipid globules have a diameter between 2 and 12 μm. In a more preferred embodiment, at least 45 vol. %, preferably at least 55 vol. %, more preferably at least 65 vol. %, and most preferably at least 75 vol. %, based on total lipid volume, of the lipid globules have a diameter between 2 and 10 μm. In an even more preferred embodiment, at least 45 vol. %, more preferably at least 55 vol. %, yet even more preferably at least 65 vol. %, and most preferably at least 75 vol. %, based on total lipid volume, of the lipid globules have a diameter between 4 and 10 μm. Preferably less than 5 vol. %, based on total lipid volume, of the lipid globules have a diameter above 12 μm.

Standard infant formulas, follow-on formulas or young child formulas typically have lipid globules with a mode diameter, based on volume, of about 0.3-0.5 μm and/or less than 45 vol. %, based on total lipid volume, of the lipid globules have a diameter above 2 μm.

The volume percentage of lipid globules is based on volume of total lipid. The mode diameter relates to the diameter which is the most present based on volume % of total lipid, or the peak value in a graphic representation, having on the X-as the diameter and on the Y-as the volume %.

The volume of the lipid globule and its size distribution can suitably be determined using a particle size analyzer such as a Mastersizer 2000 (Malvern Instruments, Malvern, UK), for example by the method described in Michalski et al, 2001, Lait 81:787-796.

### Phospholipid

The nutritional composition comprises 0.5 to 20 wt. % phospholipid based on total lipid, preferably 0.5 to 10 wt. %, even more preferably 0.75 to 8 wt. %, even more preferably 1.2 to 8 wt. %, and most preferably 1.5 to 5 wt. % phospholipid based on total lipid.

The lipid globules are at least partly coated on the surface with phospholipids. By ‘coating’ is meant that the outer surface layer of the lipid globules comprises phospholipid, whereas phospholipid is virtually absent in the core of the lipid globule. A suitable way to determine whether phospholipid is located on the surface of lipid globules is confocal laser scanning microscopy or transmission electron microscopy; see for instance Gallier et al. (A novel infant milk formula concept: Mimicking the human milk fat globule structure, Colloids and Surfaces B: Biointerfaces 136 (2015) 329-339).

The nutritional composition preferably comprises glycerophospholipids. Examples of glycerophospholipids are phosphatidylcholine (PC), phosphatidylserine (PS), phosphatidylethanolamine (PE), phosphatidylinositol (PI) and phosphatidylglycerol (PG). Preferably the nutritional composition comprises one or more of PC, PS, PI and PE, more preferably the nutritional composition comprises at least PC.

The nutritional composition preferably comprises sphingomyelin. Sphingomyelins have a phosphorylcholine or phosphorylethanolamine molecule esterified to the 1-hydroxy group of a ceramide. They are classified as phospholipid as well as sphingolipid, but are not classified as a glycerophospholipid nor as a glycosphingolipid. Preferably the nutritional composition comprises 0.05 to 10 wt. % sphingomyelin based on total lipid, more preferably 0.1 to 5 wt. %, even more preferably 0.2 to 2 wt. % based on total lipid. Preferably the nutritional composition comprises at least 5 wt. %, more preferably 5 to 40 wt. % sphingomyelin based on total phospholipid, more preferably 10 to 35 wt. %, even more preferably 15 to 35 wt. %, based on total phospholipid.

The nutritional composition preferably comprises glycosphingolipids. Preferably the nutritional composition comprises 0.1 to 10 wt. % glycosphingolipids based on total lipid, more preferably 0.5 to 5 wt. %, even more preferably 2 to 4 wt. %, based on total lipid. The term glycosphingolipids in the present context particularly refers to glycolipids with an amino alcohol sphingosine. The sphingosine backbone is O-linked to a charged head-group such as ethanolamine, serine or choline backbone. The backbone is also amide linked to a fatty acyl group. Glycosphingolipids are ceramides with one or more sugar residues joined in a beta-glycosidic linkage at the 1-hydroxyl position, and include gangliosides. Preferably the nutritional composition contains gangliosides, more preferably at least one ganglioside selected from the group consisting of GM3 and GD3.

The nutritional composition preferably comprises phospholipid derived from mammalian milk. Preferably the nutritional composition comprises phospholipid and glycosphingolipid derived from mammalian milk. The nutritional composition preferably comprises phospholipid and optionally glycosphingolipid from mammalian milk from cows, mares, sheep, goats, buffalos, horses and/or camels. More preferably the nutritional composition comprises phospholipid and optionally glycosphingolipid from cow's milk.

Phospholipid derived from milk includes preferably phospholipid that is isolated from milk fat, cream lipid, cream serum lipid, butter serum lipid (beta serum lipid), whey lipid, cheese lipid and/or buttermilk lipid. Buttermilk lipid is typically obtained during the manufacture of buttermilk. Butter serum lipid or beta serum lipid is typically obtained during the manufacture of anhydrous milk fat from butter. Preferably the phospholipid and optionally glycosphingolipid is obtained from milk cream. Examples of suitable commercially available sources for phospholipid from milk are BAEF, SM2, SM3 and SM4 powder of Corman, Salibra of Glanbia, Lipamin M20 of Lecico, Vivinal® MFGM of FrieslandCampina and LacProdan MFGM-10 or PL20 of Arla.

The use of phospholipid from milk fat advantageously comprises the use of milk fat globule membranes, which are more reminiscent to the situation in human milk. The concomitant use of phospholipid derived from milk and triglycerides derived from a mix of vegetable lipid and mammalian milk fat therefore enables the manufacture of coated lipid globules with a coating more similar to human milk, while at the same time providing an optimal fatty acid profile. Preferably the phospholipid is derived from mammalian milk fat, more preferably from cow's mammalian milk fat. Preferably the phospholipid is derived from or forms part of the milk fat globule membrane (MFGM), more preferably is derived from or forms part of cow's MFGM. Preferably the nutritional composition comprises phospholipid and glycosphingolipid. In a preferred embodiment the weight ratio of phospholipid:glycosphingolipid is from 2:1 to 12:1, more preferably from 2:1 to 10:1 and even more preferably 2:1 to 5:1.

Methods for obtaining lipid globules with an increased size and coating with phospholipid are for example disclosed in WO 2010/027258 and WO 2010/027259.

### Digestible Carbohydrates

The nutritional composition comprises digestible carbohydrates. The digestible carbohydrates preferably provide 25 to 75% of the total calories of the nutritional composition. Preferably the digestible carbohydrates provide 40 to 60% of the total calories. Based on calories the nutritional composition preferably comprises of 5 to 20 g of digestible carbohydrates per 100 kcal, more preferably 6 to 16 g per 100 kcal. When in liquid form, e.g. as a ready-to-feed liquid, the nutritional composition preferably comprises 3 to 30 g digestible carbohydrate per 100 ml, more preferably 6 to 20 g, even more preferably 7 to 10 g per 100 ml. Based on dry weight the nutritional composition preferably comprises 20 to 80 wt. %, more preferably 40 to 65 wt. % of digestible carbohydrates.

Preferred digestible carbohydrate sources are one or more of lactose, glucose, sucrose, fructose, galactose, maltose, starch and maltodextrin. Lactose is the main digestible carbohydrate present in human milk. Lactose has a low glycaemic index. The nutritional composition preferably comprises lactose. The nutritional composition preferably comprises digestible carbohydrate, wherein at least 35 wt. %, more preferably at least 50 wt. %, more preferably at least 75 wt. %, even more preferably at least 90 wt. %, most preferably at least 95 wt. % of the digestible carbohydrate is lactose. Based on dry weight the nutritional composition preferably comprises at least 25 wt. % lactose, preferably at least 40 wt. % lactose.

### Protein

The nutritional composition comprises protein. The protein preferably provides 5 to 20% of the total calories. Preferably the nutritional composition comprises protein that provides 6 to 12% of the total calories. Preferably the nutritional composition comprises less than 3.5 g protein per 100 kcal, more preferably the nutritional composition comprises between 1.5 and 2.1 g protein per 100 kcal, even more preferably between 1.6 and 2.0 g protein per 100 kcal. A low protein concentration advantageously is closer to human milk as human milk comprises a lower amount of protein based on total calories compared to cow's milk. The protein concentration in a nutritional composition is determined by the sum of protein, peptides and free amino acids. Based on dry weight the nutritional composition preferably comprises less than 12 wt. % protein, more preferably between 9.6 and 12 wt. %, even more preferably between 10 and 11 wt. %. Based on a ready-to-drink liquid product the nutritional composition preferably comprises less than 1.5 g protein per 100 ml, more preferably between 1.2 and 1.5 g per 100 ml, even more preferably between 1.25 and 1.35 g per 100 ml.

The source of the protein is preferably selected in such a way that the minimum requirements for essential amino acid content are met and satisfactory growth is ensured. Hence protein sources based on cows' milk proteins such as whey, casein and mixtures thereof and proteins based on soy, potato or pea are preferred. In case whey proteins are used, the protein source is preferably based on acid whey or sweet whey, whey protein isolate or mixtures thereof. Preferably the nutritional composition comprises at least 3 wt. % casein based on dry weight. Preferably the casein is intact and/or non-hydrolyzed.

### Non-Digestible Carbohydrates

The nutritional composition preferably comprises non-digestible oligosaccharides. Preferably the nutritional composition comprises non-digestible oligosaccharides with a degree of polymerization (DP) between 2 and 250, more preferably between 3 and 60.

Preferably the nutritional composition comprises fructo-oligosaccharides, galacto-oligosaccharides and/or galacturonic acid oligosaccharides, more preferably fructo-oligosaccharides and/or galacto-oligosaccharides, even more preferably galacto-oligosaccharides, most preferably transgalacto-oligosaccharides. In a preferred embodiment the nutritional composition comprises a mixture of galacto-oligosaccharides and fructo-oligosaccharides, more preferably transgalacto-oligosaccharides and fructo-oligosaccharides. Suitable non-digestible oligosaccharides are for example Vivinal®GOS (FrieslandCampina DOMO), Raftilin®HP or Raftilose® (Orafti).

Preferably, the nutritional composition comprises 80 mg to 2 g non-digestible oligosaccharides per 100 ml, more preferably 150 mg to 1.5 g per 100 ml, even more preferably 300 mg to 1 g per 100 ml. Based on dry weight, the nutritional composition preferably comprises 0.25 wt. % to 20 wt. %, more preferably 0.5 wt. % to 10 wt. %, even more preferably 1.5 wt. % to 7.5 wt. % of non-digestible oligosaccharides.

### Formula

The use according to the present invention requires the administration of an infant formula, a follow-on formula or a young child formula. This means that the nutritional composition is not human milk. It also means that the nutritional composition is not native cow's milk or native milk from another mammal. In the context of the present invention, young child formula can also be named growing-up milk.

Alternatively, the terms as used herein, “infant formula” or “follow-on formula” or “young child formula” means that it concerns a composition that is artificially made or in other words that it is synthetic. Hence in one embodiment, the nutritional composition that is administered is an artificial infant formula or an artificial follow-on formula or an artificial young child formula or a synthetic infant formula or a synthetic follow-on formula or a synthetic young child formula.

In the present invention, infant formula refers to nutritional compositions, artificially made, intended for infants of 0 to about 4 to 6 months of age and are intended as a substitute for human milk. Typically, infant formulae are suitable to be used as sole source of nutrition. Such infant formulae are also known as starter formula. Follow-on formula for infants starting with at 4 to 6 months of life to 12 months of life are intended to be supplementary feedings for infants that start weaning on other foods. Infant formulae and follow-on formulae are subject to strict regulations, for example for the EU regulations no. 609/2013 and no. 2016/127. In the present context, young child formula refers to nutritional compositions, artificially made, intended for infants of 12 months to 36 months, which are intended to be supplementary feedings for infants.

The nutritional composition is preferably an infant formula or a follow-on formula. More preferably the nutritional composition is an infant formula.

The nutritional composition comprises digestible carbohydrates, protein and lipid, wherein the lipid preferably provides 30 to 60% of the total calories, the protein provides 5 to 20% of the total calories and the digestible carbohydrates provide 25 to 75% of the total calories.

The nutritional composition is preferably an infant formula or follow-on formula and preferably comprises 3 to 7 g lipid/100 kcal, preferably 4 to 6 g lipid/100 kcal, more preferably 4.5 to 5.5 g lipid/100 kcal, preferably comprises 1.7 to 3.5 g protein/100 kcal, more preferably 1.8 to 2.1 g protein/100 kcal, more preferably 1.8 to 2.0 g protein/100 kcal and preferably comprises 5 to 20 g digestible carbohydrate/100 kcal, preferably 6 to 16 g digestible carbohydrate/100 kcal, more preferably 10 to 15 g digestible carbohydrate/100 kcal.

Preferably the nutritional composition is an infant formula or follow-on formula, and preferably has an energy density of 60 to 75 kcal/100 ml, more preferably 60 to 70 kcal/100 ml, when in a ready-to-drink form. This density ensures an optimal balance between hydration and caloric intake.

In one embodiment, the nutritional composition is a powder. Suitably, the nutritional composition is in a powdered form, which can be reconstituted with water or other food grade aqueous liquid, to form a ready-to drink liquid, or is in a liquid concentrate form that should be diluted with water to a ready-to-drink liquid. It was found that lipid globules maintained their size and coating when reconstituted.

A second aspect of the invention relates to a nutritional composition, selected from infant formula, follow-on formula and young child formula, comprising digestible carbohydrates, protein and lipid, wherein the lipid is in the form of lipid globules and wherein
a. the lipid globules have a mode diameter, based on volume, of at least 1.0 μm; and/or
b. at least 45 volume %, based on total lipid volume, of the lipid globules have a diameter of 2 to 12 μm; and
c. the lipid comprises 0.5 to 20 wt. % phospholipids based on total lipids and wherein the lipid globules are at least partly coated on the surface with phospholipids,
   for use in increasing the levels of myelin-associated glycoprotein (MAG), myelin basic protein (MBP), or combinations thereof in the brain of an infant who has delayed brain development or is at risk of having delayed brain development.

A third aspect of the invention relates to a nutritional composition, selected from infant formula, follow-on formula and young child formula, comprising digestible carbohydrates, protein and lipid, wherein the lipid is in the form of lipid globules and wherein
a. the lipid globules have a mode diameter, based on volume, of at least 1.0 μm; and/or
b. at least 45 volume %, based on total lipid volume, of the lipid globules have a diameter of 2 to 12 μm; and
c. the lipid comprises 0.5 to 20 wt. % phospholipids based on total lipids and wherein the lipid globules are at least partly coated on the surface with phospholipids,
   for use in increasing the brain cell membrane levels of palmitic acid (C16:0), stearic acid (C18:0), or combinations thereof, in the brain of an infant who has delayed brain development or is at risk of having delayed brain development.

The embodiments described herein before with regards to the first aspect of the invention, preferably also apply to the second and third aspect of the invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Experimental Diets Used in Example 1 and 2

The offspring diets (Control or Test) were provided to the animals in the form of soft dough that was freshly prepared on a daily base. The soft dough was placed on the cage floor to allow easy access for the animals.

The offspring diets comprised a macronutrient and micronutrient composition following AlN93G (Reeves P G, et al., AlN-93 purified diets for laboratory rodents: final report of the American Institute of Nutrition ad hoc writing committee on the reformulation of the AlN-76A rodent diet. J Nutr 1993; 123:1939-51). The offspring diets consisted of 28.3% (w/w) infant milk formula (IMF). The fat components were derived entirely from the IMF. Protein, carbohydrates, and micronutrients were added to match AlN93G. Per experiment the fat content and fatty acid profile of the Control and Test diets were kept the same.

TABLE 1
Fatty acid composition (g/100 g IMF powder)

| | Example 1 | | Example 2 | |
|---|---|---|---|---|
| | Control 1 | Test 1 | Control 2 | Test 2 |
| Total fatty acids | 23.5 | 23.5 | 23.2 | 22.7 |
| Saturated fatty acids | 9.80 | 9.85 | 10.4 | 10.1 |
| Monounsaturated fatty acids | 9.13 | 9.35 | 8.89 | 8.74 |
| Linoleic acid | 3.22 | 3.31 | 3.19 | 3.17 |
| Alpha linolenic acid | 0.59 | 0.59 | 0.59 | 0.59 |
| Arachidonic acid | 0.09 | 0.09 | 0.08 | 0.08 |
| Docosahexaenoic acid | 0.08 | 0.08 | 0.05 | 0.05 |
| Total omega-6 PUFA | 3.32 | 3.42 | 3.29 | 3.27 |
| Total omega-3 PUFA | 0.69 | 0.69 | 0.66 | 0.65 |

### Test IMF

The test IMF's was prepared in a similar way as described in example 1B of WO 2010/0027259. The lipid globules of Test IMF's were large and coated with phospholipids. Test 1 IMF comprised phospholipids derived from egg yolk. Test 2 IMF comprised beta-serum powder as a source of milk derived phospholipids including parts of milk fat globule membrane (MFGM). The phospholipid source was added before homogenization, which resulted in phospholipids being present in the coating of the lipid globules.

### Control IMF

The Control IMF's were prepared in the conventional way with a high pressure homogenization, resulting in small lipid globules. The Control IMF's comprised the same source and same amount of phospholipids as the Test IMF's. The phospholipid sources were added after homogenization and were therefor not located in the coating of the lipid globules.

Size distribution of the lipid droplets was determined by laser light-scattering analysis (Mastersizer 2000, Hydro 200G, Malvern Instruments Limitedy, Worcestershire, UK). The detailed characteristics of the lipid globule structure in the different IMF's are shown in Table 2.

TABLE 2

| | Example 1 | | Example 2 | |
|---|---|---|---|---|
| | Control 1 | Test 1 | Control 2 | Test 2 |
| Lipid globule size - volume- | 0.4 | 3.2 | 0.5 | 4.8 |
| weighted mode diameter | | | | |
| Volume-based percentage of | 16.0 | 47.5 | 4.3 | 60.7 |
| lipid globules having a | | | | |
| diameter between 2 and | | | | |
| 12 μm | | | | |
| Phospholipid source | Egg phospholipid | | Milk MFGM | |
| | (egg yolk) | | (beta-serum) | |
| Phospholipids (wt. % based | 1.8 | 1.8 | 1.64 | 1.68 |
| on total lipid) | | | | |
| Phospholipids in the coating | No | No | No | Yes |

### Example 1

Mouse dams were exposed to an omega 3 deficient diet during pregnancy and lactation, resulting in impaired brain development in the offspring (Carrie et al., 2000, DOI:https://doi.org/10.1016/S0022-2275(20)34486-2). C57BL/6J mice were used in this experiment. From 4 weeks prior to breeding, the female mice were kept on a semisynthetic AlN-93G based rodent diet with 7% (w/w) total fat by weight of the diet, which included 2.58% (w/w) linoleic acid C18:2 (n6) and 0.01% (w/w) alpha linoleic acid C18:3 (n3) by weight of the diet as source of omega 6 and omega 3 PUFA's respectively (=omega 3-deficient diet). The omega 3-deficient diet was continued throughout pregnancy and lactation. The day that the litter was born was considered postnatal day (PN) 0. At PN2, litters were culled to 6 pups per dam (litters containing at least 2 males and 2 females). From PN16 onwards litters (with dam) were randomly assigned to receive either of the two experimental diets. At PN21 the male offspring was weaned and their respective diets were continued. At PN42, the mice offspring were sacrificed. The brains were harvested, the hippocampi were dissected.

Of each brain, 1 hippocampus was homogenized (Utra-Turrax T25 basic, IKA, VWR international) in 50 volumes of ice cold deionized water (MiliQ). Subsequently, brain fatty acid (FA) profile was quantified by means of gas chromatographic analysis. 1 ml brain homogenate was extracted according to the procedure of Bligh & Dyer (dichloromethane/methanol extraction). The lipids were converted into methyl esters with concentrated sulfuric acid in methanol. The fatty acid methyl esters (FAME) were extracted from the methanol solution with hexane and analyzed on a gas chromatograph (GC) equipped with a flame ionization detector (FID).

The other hippocampus per animal was used to quantify gene expression (qPCR) of Myelin Basic Protein (MBP) and Myelin-Associated Glycoprotein (MAG), which are markers for myelin. Total RNA was isolated using the RNeasy mini kit (©QIAGEN) followed by cDNA synthesis by use of the iScript™ cDNA Synthesis Kit (Bio-rad). Obtained cDNA was used for qPCR to quantify gene expression of MBP and MAG with use of the SYBR™ Select Master Mix (Applied Biosystems™).

All data was analyzed with SPSS 19.0 software (SPSS Benelux, Gorinchem, The Netherlands) using student T-tests. Individual animals were regarded as experimental units.

### Results

Brain fatty acid species were expressed as a percentage of total fatty acids (% FA) in Table 3. The values between brackets are the standard deviation.

TABLE 3

| | Control 1 | Test 1 |
|---|---|---|
| | (n = 10) | (n = 12) |
| C16:0 (palmitic acid) | 22.95 (0.14) | 23.33 (0.15) \hat{ } |
| C18:0 (stearic acid) | 21.12 (0.08) | 21.43 (0.09) * |

\hat{ } p < 0.1 vs. Control
* p < 0.05 vs. Control

The fatty acids palmitic acid (C16:0) and stearic acid (C18:0) are the main saturated fatty acids in brain tissue. Brain myelin in particular is rich in these fatty acids. Both C16:0 and C18:0 were increased in the brains of offspring fed with the Test 1 diet compared to the brains of offspring fed with Control 1.

Gene expression (relative to expression of housekeeping genes 18s, B2M, Gusb, HRPT1, TBP, Tubb3 and actin) of hippocampal MAG and MBP was higher in the brains of offspring fed Test 1 than in the brains of offspring fed with Control 1. See Table 4 below. The values between brackets are the standard error of the mean.

TABLE 4

| | Control 1 | Test 1 |
|---|---|---|
| | (n = 10) | (n = 12) |
| MAG | 0.98 (0.10) | 1.36 (0.17) * |
| MBP | 0.92 (0.12) | 1.29 (0.18) \hat{ } |

\hat{ } p < 0.1 vs. Control
* p < 0.05 vs. Control

### Example 2

C57BL/6J mice were used in this experiment. The day that the litter was born was considered postnatal day (PN) 0. At PN2, litters were randomized and culled to a standardized litter size of 3 pups per dam (litters containing at least 1 male and 1 female). From PN16 onwards litters (with dam) were randomly assigned to receive either of the two experimental diets. After 5 days of diet exposure, thus at PN21, mice offspring were sacrificed.

Of each brain, 1 hemisphere was homogenized (Utra-Turrax T25 basic, IKA, VWR international) in 50 volumes of ice cold deionized water (MiliQ). Subsequently, brain fatty acid (FA) profile was quantified by means of gas chromatographic analysis. 1 ml brain homogenate was extracted according to the procedure of Bligh & Dyer (dichloromethane/methanol extraction). The lipids were converted into methyl esters with concentrated sulfuric acid in methanol. The fatty acid methyl esters (FAME) were extracted from the methanol solution with hexane and analyzed on a gas chromatograph (GC) equipped with a flame ionization detector (FID).

The data was analyzed with SPSS 19.0 software (SPSS Benelux, Gorinchem, The Netherlands) using student T-tests. Individual animals were regarded as experimental units.

### Results

Brain fatty acid species were expressed as a percentage of total fatty acids (% FA) in Table 5. The values between brackets are the standard deviation.

TABLE 5

| | Control 2 | Test 2 |
|---|---|---|
| | (n = 18) | (n = 19) |
| C16:0 (palmitic acid) | 25.56 (0.12) | 26.14 (0.22) * |
| C18:0 (stearic acid) | 19.01 (0.05) | 19.36 (0.10) * |

* p < 0.05 vs. Control

Both C16:0 and C18:0 were increased in the brains of offspring fed with the Test 2 diet compared to the brains of offspring fed with Control 2.

### Example 3

Infant formula, intended for infants of 0 to 6 months of age, comprising per 100 ml, after reconstituting 13.7 g powder to an end volume of 100 ml:
66 kcal
1.3 g protein (whey protein/casein weight ratio 1/1)
7.3 g digestible carbohydrates (mainly being lactose)
3.4 gram fat (comprising by weight of total lipid 97 wt. % vegetable oil, about 1.5 wt. % of milk derived phospholipids, the remainder being fish oil and microbial oil)
0.8 g non-digestible oligosaccharides, of which 0.08 g long chain fructo-oligosaccharides (source RaftilineHP) and 0.72 g trans-galacto-oligosaccharides (source Vivinal GOS)
minerals, vitamins, trace elements and other micronutrients as according to directives for infant formula.

The formula comprises lipid globules with a volume mode diameter of about 5.6 μm and the volume % of lipid globules with a mode diameter between 2 and 12 μm is above 45.

1.-14. (canceled)

15. A method for increasing myelination in the brain of an infant who has delayed brain development or is at risk of having delayed brain development, the method comprising administering to the infant a nutritional composition comprising digestible carbohydrates, protein and lipid, wherein the lipid is in the form of lipid globules and wherein
a. the lipid globules have a mode diameter, based on volume, of at least 1.0 μm; and/or
at least 45 volume %, based on total lipid volume, of the lipid globules have a diameter of 2 to 12 μm; and
b. the lipid comprises 0.5 to 20 wt. % phospholipids based on total lipids and wherein
the lipid globules are at least partly coated on the surface with phospholipids,
wherein the nutritional composition is selected from infant formula, follow-on formula and young child formula.

16. The method according to claim 15, wherein the increased myelination contributes to improved cognitive development and/or motor function in the infant.

17. The method according to claim 15, wherein the increased myelination is around the axons connected to and within the hippocampus.

18. The method according to claim 15, wherein the infant is a child aged 0-12 months.

19. The method according to claim 15, wherein the infant who has delayed brain development or is at risk of having delayed brain development is selected from preterm infants, small-for-gestational age infants, infants with phenylketonuria (PKU), infants with epilepsy, infants with cerebral palsy, infants born with a brain injury, infants who were exposed to perinatal hypoxia, or combinations thereof.

20. The method according to claim 15, wherein the phospholipids comprise at least 5 wt. % sphingomyelin based on total phospholipids.

21. The method according to claim 15, wherein the phospholipids are derived from mammalian milk fat.

22. The method according to claim 15, wherein the lipid contains at least 10 wt. % palmitic acid based on total fatty acids and at least 15 wt. % of palmitic acid, based on total palmitic acid, is located at the sn-2 position of a triglyceride.

23. The method according to claim 15, wherein the increased myelination is by higher levels in the brain of myelin-associated glycoprotein (MAG), myelin basic protein (MBP), or combinations thereof.

24. The method according to claim 15, wherein the increased myelination is by increasing the brain cell membrane levels of palmitic acid (C16:0), stearic acid (C18:0), or combination thereof.

25. The method according to claim 15, wherein the nutritional composition is an infant formula or follow-on formula.

26. The method according to claim 15, wherein the increased myelination in the brain of the infant is compared to a similar infant who has delayed brain development or is at risk of having delayed brain development, which consumed a nutritional composition, selected from infant formula, follow-on formula and young child formula, comprising digestible carbohydrates, protein and lipid, and wherein the lipid is in the form of lipid globules and wherein:
a. the lipid globules have a mode diameter, based on volume, of around 0.5 μm, and
b. less than 45 volume %, based on total lipid volume, of the lipid globules have a diameter above 2 μm, and
c, wherein the lipid comprises less than 0.5 wt. % phospholipids based on total lipids and wherein the lipid globules are not coated with phospholipids.

27. A method for increasing the levels of myelin-associated glycoprotein (MAG), myelin basic protein (MBP), or combinations thereof in the brain of an infant who has delayed brain development or is at risk of having delayed brain development, the method comprising administering to the infant a nutritional composition comprising digestible carbohydrates, protein and lipid, wherein the lipid is in the form of lipid globules and wherein
a. the lipid globules have a mode diameter, based on volume, of at least 1.0 μm; and/or
at least 45 volume %, based on total lipid volume, of the lipid globules have a diameter of 2 to 12 μm; and
b. the lipid comprises 0.5 to 20 wt. % phospholipids based on total lipids and wherein
the lipid globules are at least partly coated on the surface with phospholipids,
wherein the nutritional composition is selected from infant formula, follow-on formula and young child formula.

28. A method for increasing the brain cell membrane levels of palmitic acid (C16:0), stearic acid (C18:0), or combinations thereof, in the brain of an infant who has delayed brain development or is at risk of having delayed brain development, the method comprising administering to the infant a nutritional composition comprising digestible carbohydrates, protein and lipid, wherein the lipid is in the form of lipid globules and wherein
a. the lipid globules have a mode diameter, based on volume, of at least 1.0 μm; and/or
at least 45 volume %, based on total lipid volume, of the lipid globules have a diameter of 2 to 12 μm; and
b. the lipid comprises 0.5 to 20 wt. % phospholipids based on total lipids and wherein
the lipid globules are at least partly coated on the surface with phospholipids,
wherein the nutritional composition is selected from infant formula, follow-on formula and young child formula.
